(19) United States
(12) Patent Application Publication
(10) Pub. No.: US 2020/0411141 A1
(43) Pub. Date: Dec. 31, 2020

(54) COMPUTER IMPLEMENTED OPHTHALMOLOGY SITE SELECTION AND PATIENT IDENTIFICATION TOOLS

(71) Applicant: VERANA HEALTH, INC., San Francisco, CA (US)

(72) Inventors: Douglas Nelson FOSTER, San Francisco, CA (US); Nikolai STEKLOV, San Francisco, CA (US); Carol HOANG, San Francisco, CA (US); Nitin KARANDIKAR, Cupertino, CA (US); Paula ALVES, San Francisco, CA (US); Cuau LEYVA, San Francisco, CA (US); Kevin WOOD, San Francisco, CA (US)

(21) Appl. No.: 16/975,733

(22) PCT Filed: Feb. 27, 2019
(86) PCT No.: PCT/US2019/019797

§ 371 Date: Aug. 26, 2020

Related U.S. Application Data
(60) Provisional application No. 62/636,038, filed on Feb. 27, 2018.

(51) Int. Cl. G16H 10/20; G16H 10/60; G16H 50/30

(52) U.S. Cl. CPC G16H 10/20; A61B 3/0025; G16H 50/30; G16H 10/60

(57) ABSTRACT

Provided herein are systems and methods for collecting, distributing, analyzing, and organizing patient data for clinical trial selection, placement, and enrollment. Automated systems and methods are provided for acquiring patient health data and clinical trial requirements, and automatically identifying and determining patients that are eligible for a clinical trial. A classifier can identify and/or output patients that may be eligible for clinical trials in the future.

### CROSS REFERENCE TO RELATED APPLICATIONS

This application claims the benefit under 35 U.S.C. 119 of U.S. Provisional Patent Application No. 62/636,038, filed Feb. 27, 2018, titled “COMPUTER IMPLEMENTED OPHTHALMOLOGY SITE SELECTION AND PATIENT IDENTIFICATION TOOLS”, which application is incorporated by reference as if fully set forth herein.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Various aspects of the invention described herein relate to methods, systems, and architectures for collection, transformation, conditioning, and correlation of patient and medical data related to a selected medical practice. In some aspects, there are provided methods, systems and architecture for assimilation of data from multiple different medical practices. Medical practices are related by one or more of membership in medical, physician or surgeon associations, provide treatment to similar patient types, provide treatment to similar disorders of the body, performs similar therapeutic, interventional or diagnostic procedures on the body, practice or enlist similar diagnostic procedures or prescribe similar pharmacological agents.

### BACKGROUND

Advancements in computer processing, data collection and analytics continue to modernize and change the practice of medicine and the structure of the healthcare system. However, a large number of these efforts have been directed to the extremes of the healthcare system. On one end, there are data systems for handling and analyzing individual patient electronic medical records (EMR), usually within an insurance plan or hospital system or systems. On the other extreme are national data bases typically directed to national government sponsored health care and associated payment and utilization data.

One area in need of improved data management and analytics is in the specialized medical practice.

### SUMMARY OF THE DISCLOSURE

Disclosed herein is a method of identifying patients for enrollment in a clinical trial, the method comprising the steps of acquiring, in a computing device, clinical trial data including eligibility requirements for the clinical trial, acquiring, in the computing device, patient health data for a plurality of patients, determining, in the computing device, a subset of patients from the plurality of patients that are eligible for the clinical trial based on the clinical trial data and the patient health data, and outputting from the computing device the subset of patients.

In some examples, the acquiring clinical trial data step comprises acquiring clinical trial data from a remote database.

In some examples, the eligibility requirements comprise a disease type, disease progression, symptoms, treatment type, age, gender, geographic location, proximity to clinical sites, and outcomes.

In one example, the acquiring patient health data step comprises acquiring patient health data from a remote database, such as the IRIS registry.

In some examples, the patient health data comprises patient health, disease state, age, gender, geographic location, treatment type, symptoms, disease progression, other relevant diseases, physician notes, and billing codes relating to disease diagnosis and treatment.

In one example, the method can further comprise assigning an eligibility score to each of the plurality of patients based on how many eligibility requirements are satisfied by each patient.

A non-transitory computing device readable medium having instructions stored thereon for identifying patients for enrollment in a clinical trial, wherein the instructions are executable by a processor to cause a computing device to acquire clinical trial data including eligibility requirements for the clinical trial, acquire patient health data for a plurality of patients, determine a subset of patients from the plurality of patients that are eligible for the clinical trial based on the clinical trial data and the patient health data, and output the subset of patients.

In one example, the instructions are executable by the processor to cause the computing device to acquire clinical trial data from a remote database.

In some examples, the eligibility requirements comprise a disease type, disease progression, symptoms, treatment type, age, gender, geographic location, proximity to clinical sites, and outcomes.

In one example, the instructions are executable by the processor to cause the computing device to acquire patient health data from a remote database, such as from an IRIS registry.

In some examples, the patient health data comprises patient health, disease state, age, gender, geographic location, treatment type, symptoms, disease progression, other relevant diseases, physician notes, and billing codes relating to disease diagnosis and treatment.

In one example, the instructions are executable by the processor to cause the computing device to assign an eligibility score to each of the plurality of patients based on how many eligibility requirements are satisfied by each patient.

Also disclosed is a method of identifying patients for enrollment in a clinical trial, the method comprising the steps of acquiring, in a computing device, clinical trial data including eligibility requirements and a start date for the clinical trial, acquiring, in the computing device, patient health data for a plurality of patients, applying the clinical trial data and the patient health data to a classifier of the computing device to identify a subset of patients from the plurality of patients that are not currently eligible for the clinical trial but will likely be eligible for the clinical trial in the future, and outputting the subset of patients from the computing device.

In some examples, the acquiring clinical trial data step comprises acquiring clinical trial data from a remote database.

In some examples, the eligibility requirements comprise a disease type, disease progression, symptoms, treatment type, age, gender, geographic location, proximity to clinical sites, and outcomes.

In one example, the acquiring patient health data step comprises acquiring patient health data from a remote database, such as the IRIS registry.

In some examples, the patient health data comprises patient health, disease state, age, gender, geographic location, treatment type, symptoms, disease progression, other relevant diseases, physician notes, and billing codes relating to disease diagnosis and treatment.

In one example, the method can further comprise assigning an eligibility score to each of the plurality of patients based on how many eligibility requirements are satisfied by each patient.

In some examples, each of the subset of patients are assigned a binary score indicating that a patient is likely to be eligible for the clinical trial in the future. In other examples, each of the subset of patients are assigned a linear score indicating along a scale how likely a patient is likely to be eligible for the clinical trial in the future.

A non-transitory computing device readable medium having instructions stored thereon for identifying patients for enrollment in a clinical trial, wherein the instructions are executable by a processor to cause a computing device to acquire clinical trial data including eligibility requirements and a start date for the clinical trial, acquire patient health data for a plurality of patients, apply the clinical trial data and the patient health data to a classifier of the computing device to identify a subset of patients from the plurality of patients that are not currently eligible for the clinical trial but will likely be eligible for the clinical trial in the future, and output the subset of patients.

In one example, the instructions are executable by the processor to cause the computing device to acquire clinical trial data from a remote database.

In some examples, the eligibility requirements comprise a disease type, disease progression, symptoms, treatment type, age, gender, geographic location, proximity to clinical sites, and outcomes.

In one example, the instructions are executable by the processor to cause the computing device to acquire patient health data from a remote database, such as from an IRIS registry.

In some examples, the patient health data comprises patient health, disease state, age, gender, geographic location, treatment type, symptoms, disease progression, other relevant diseases, physician notes, and billing codes relating to disease diagnosis and treatment.

In one example, the instructions are executable by the processor to cause the computing device to assign an eligibility score to each of the plurality of patients based on how many eligibility requirements are satisfied by each patient.

In some examples, each of the subset of patients are assigned a binary score indicating that a patient is likely to be eligible for the clinical trial in the future. In another example, each of the subset of patients are assigned a linear score indicating along a scale how likely a patient is likely to be eligible for the clinical trial in the future.

A method of identifying patients for enrollment in a clinical trial is also provided, the method comprising the steps of receiving, in a computing device, inclusion/exclusion criteria for the clinical trial, acquiring, in the computing device, patient health data for a plurality of patients, determining, in the computing device, a list of patients that are eligible for the clinical trial, determining, in the computing device, a geographic location that includes a plurality of eligible patients proximate to a clinical site, and outputting from the computing device the geographic location, the plurality of eligible patients, and the clinical site.

In some examples, the inclusion/exclusion criteria are received from a user.

In one example, the inclusion/exclusion criteria comprise a disease type, disease progression, symptoms, treatment type, age, gender, geographic location, proximity to clinical sites, and outcomes.

In some examples, patient health data step is acquired from a remote database, such as an IRIS registry.

In some examples, the patient health data comprises patient health, disease state, age, gender, geographic location, treatment type, symptoms, disease progression, other relevant diseases, physician notes, and billing codes relating to disease diagnosis and treatment.

In one example, the determining step further comprises assigning an eligibility score to each of the plurality of patients based on how many inclusion/exclusion criteria are satisfied by each patient.

Also provided is a non-transitory computing device readable medium having instructions stored thereon for identifying patients for enrollment in a clinical trial, wherein the instructions are executable by a processor to cause a computing device to receive inclusion/exclusion criteria for the clinical trial, acquire patient health data for a plurality of patients, determine a list of patients that are eligible for the clinical trial, determine a geographic location that includes a plurality of eligible patients proximate to a clinical site, and output the geographic location, the plurality of eligible patients, and the clinical site.

In some examples, the inclusion/exclusion criteria comprise disease type, disease progression, symptoms, treatment type, age, gender, geographic location, proximity to clinical sites, and outcomes.

In one example the instructions are executable by the processor to cause the computing device to acquire patient health data from a remote database, such as an IRIS registry.

In some examples, the patient health data comprises patient health, disease state, age, gender, geographic location, treatment type, symptoms, disease progression, other relevant diseases, physician notes, and billing codes relating to disease diagnosis and treatment.

In one example, the instructions are executable by the processor to cause the computing device to assign an eligibility score to each of the plurality of patients based on how many inclusion/exclusion criteria are satisfied by each patient.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a diagram showing an example of a computing environment configured to configured to facilitate collecting, distributing, analyzing, and organizing patient data for clinical trial selection, placement, and enrollment.

FIG. 1B is a diagram showing an example of clinical trial engine(s).

FIG. 1C is a diagram showing an example of a patient selection engine(s).

FIG. 1D is a diagram showing an example of a future eligibility engine(s).

FIG. 1E is a diagram showing an example of a patient follow-up engine(s).

FIGS. 2A-2G illustrate examples of exemplary data visualization for a clinical trial coordinator at a specific medical practice enabled by accessing the medical practice specific data.

FIG. 3 is a flowchart illustrating one example of a process by which a medical practice can review and enroll patients in a clinical trial.

FIG. 4A is a diagram showing an example of a computing environment configured to facilitate collecting, analyzing, and reviewing patient data for clinical trial selection, placement, and enrollment.

FIG. 4B is a diagram showing an example of the patient data engine(s).

FIG. 4C is a diagram showing an example of the inclusion/exclusion engine(s).

FIGS. 5A-5D are exemplary data visualization for a clinical program manager workflow enabled by accessing medical practice specific data.

FIG. 6 is a flowchart illustrating one example of a process by which a medical practice can review and enroll patients in a clinical trial.

### DETAILED DESCRIPTION

Various aspects of the invention described herein relate to methods, systems, and architectures for collection, transformation, conditioning, and correlation of patient and medical data related to a selected medical practice. In some aspects, there are provided methods, systems and architecture for assimilation of data from multiple different medical practices. Medical practices are related by one or more of membership in medical, physician or surgeon associations, provide treatment to similar patient types, provide treatment to similar disorders of the body, performs similar therapeutic, interventional or diagnostic procedures on the body, practice or enlist similar diagnostic procedures or prescribe similar pharmacological agents.

In one aspect, the medical data collected from a specific medical practice relates to the collection of data from medical practices having physicians, surgeons, health care practitioners or health care professionals that are members of the same medical association, academy or organization. Additionally or alternatively, there may be data collected from specific medical practices having health care practitioners such as physicians, surgeons and health care professionals within a common or related medical field. Examples of medical fields include ophthalmology, optometry, orthopedics, pediatrics, oncology, bariatrics, emergency medicine, geriatrics, diagnostic radiology, psychiatry, anesthesiology, orthopedic surgery, internal medicine, medical genetics, pathology and anatomy, neurology, allergy and immunology, thoracic surgery, plastic surgery, otolaryngology, physical medicine and rehabilitation, general surgery, urology, dermatology, urological surgery, obstetrics and gynecology, family medicine, family practice, transplant hepatology, vascular and coronary surgery, medical genetics, medical microbiology, medical toxicology, interventional radiology, anesthesiology, or molecular genetic pathology.

In one specific example, the organization of related clinical practices is the American Academy of Ophthalmology (AAO) and the database of collected specific medical practice information is the IRIS Registry. In one aspect, the methods and systems described herein may address or provide products addressing the needs of life science companies as related to specific clinical practices or medical specialties. In one specific application, there are provided systems and methods for assessing data sets from multiple sources and of different types collected from multiple different medical practice groups containing real world clinical data along within payment, reimbursement and other related health insurance information from both private and public health insurance payers. As a result, in one aspect, the systems and tools described herein may be utilized with advantage to provide clinical optimization as well as market research insights across an array of clinical and real world scenarios related to the specific medical practice group under consideration. In one specific aspect, the data accessed by the systems and methods described herein relate to ophthalmology practices and patients including: demographics, visual acuity, clinical visit information, inter ocular pressure (TOP), treatments and pharmacological agents prescribed or utilized, ocular comorbidities, procedures performed as well as any of the above or other information related to eye selectivity such as both eyes, the left eye, the right eye or one eye.

In various aspects and embodiments, there is provided an overall data architecture for an exemplary specific medical practice data system. The medical practice centered data sources, collection and architecture may include medical practice collection and reporting, medical practice organization architecture and the DigiSight architecture. Still further, there may also be provide a variety of data collection and processing techniques to produce analytics for a medical practice within the medical practice specific data set. In some aspects there may be provided a specific medical practice architecture including, for example, data of the specific medical practice related to examinations, assessments, diagnostics for disorders and treatments of the eyes in an ophthalmology practice. In still further aspects, there may be provided exemplary data collection and processing methods to produce data analytics and results for specific medical practice. In an exemplary specific medical practice architecture, there may be both registry information and multiple specific medical practice data sets provided into a data lake. Additionally, there is provided data quality processes including a data pipeline to validate and enhance the data used by the systems and methods herein.

Described herein are apparatuses (e.g., systems, computing device readable media, devices, etc.) and methods for collecting, distributing, analyzing, and organizing patient data for clinical trial selection, placement, and enrollment. One object of the present disclosure is to provide tools that enable physicians and/or clinical trials coordinators to review clinical trial overviews, review identified patient lists, and select patients for enrollment. It is another object of the present disclosure to provide tools that enable life science companies to access nationwide patient databases, filter potential clinical trial enrollees with protocol optimization, and identify location(s) with the proper site location and/or patient population for clinical trials.

Another object of the present disclosure uses machine learning technology to automatically assess whether a patient that is not currently eligible for a selected clinical trial may be eligible in the future or by the start date of the clinical trial. The machine learning technology can make this determination based upon patient data including patient demographics, historical patient data, disease progression, treatment type, physician type, age, etc. These methods and apparatus can use this information to provide patient, physician, or the like. When the system is triggered by a request for patients that may be eligible for a clinical trial in the future, the system can retrieve relevant patient data from a local or remote database, process the information, and pass the information into a classifier, which may use machine learning technology (e.g., Convolutional Neural Network (CNN), Decision Tree, Random Forest, Logistic Regression, Support Vector Machine, AdaBOOST, K-Nearest Neighbor (KNN), Quadratic Discriminant Analysis, Neural Network, etc.) to return an eligibility score. The parameters inputted into the classification model can be optimized with historic data. The scoring classification model may be used to provide an output indicating the likelihood that a patient may be eligible for a future clinical trial. The results may be provided on demand and/or may be stored in a memory (e.g., database) for later use.

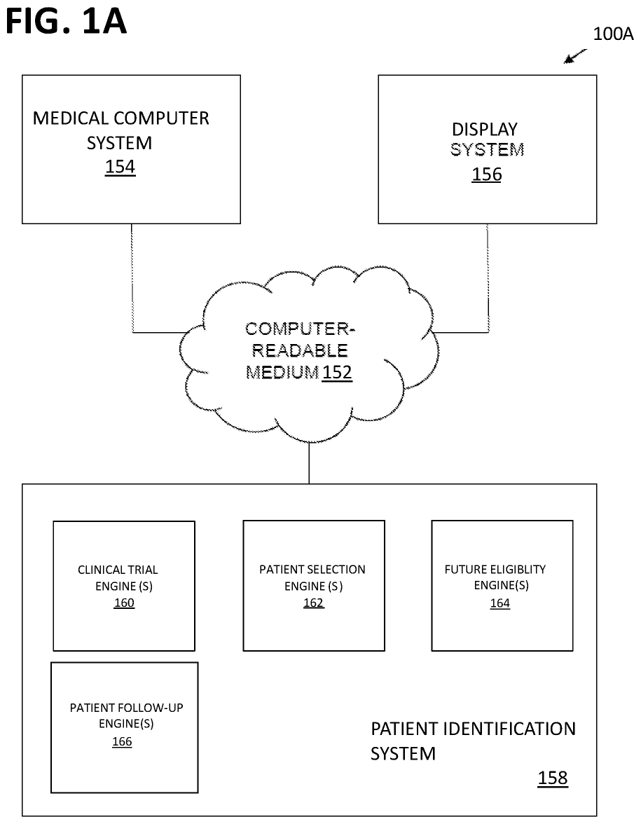

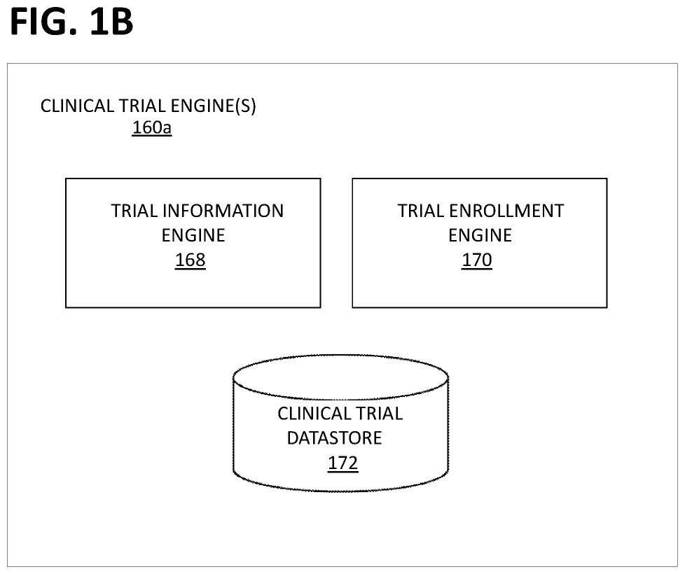

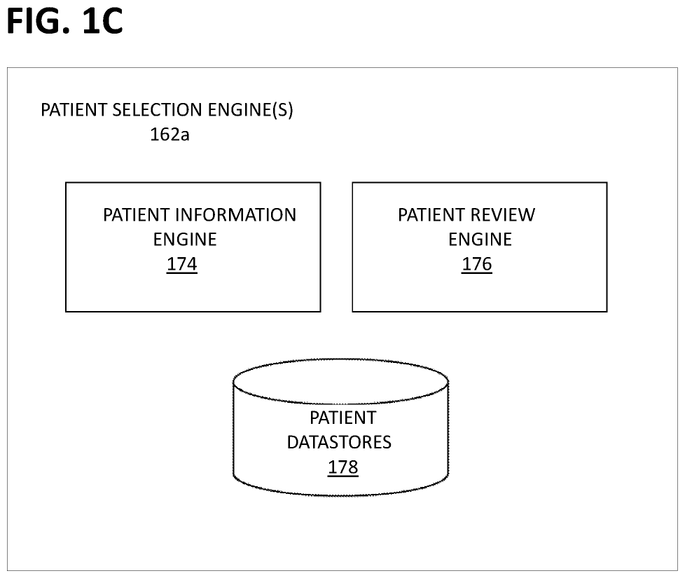

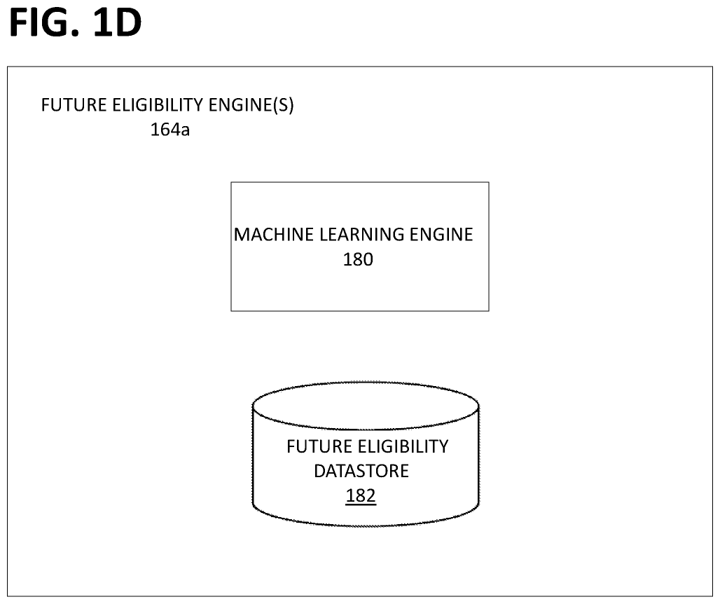

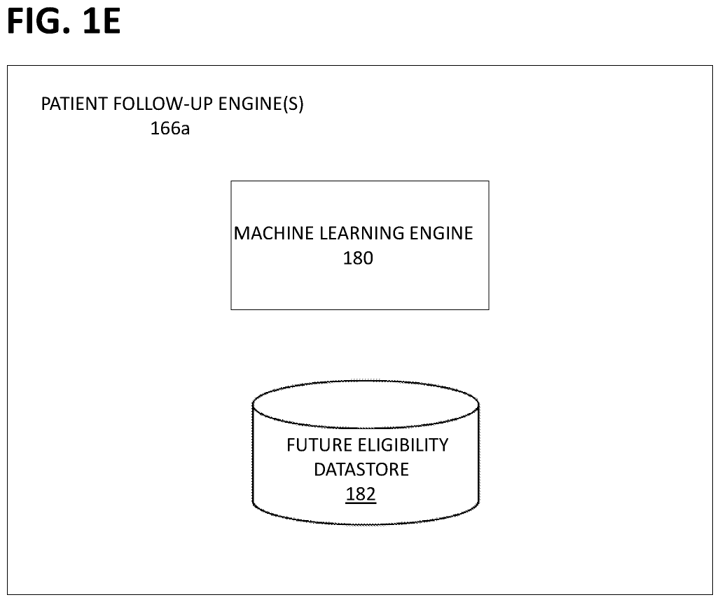

FIG. 1A is a diagram showing an example of a computing environment 100A configured to facilitate collecting, distributing, analyzing, and organizing patient data for clinical trial selection, placement, and enrollment. The environment 100A includes a computer-readable medium 152, a medical computer system 154, a display system 156, and a patient identification system 158. One or more of the modules in the computing environment 100A may be coupled to one another or to modules not explicitly shown.

The computer-readable medium 152 and other computer readable media discussed herein are intended to represent a variety of potentially applicable technologies. For example, the computer-readable medium 152 can be used to form a network or part of a network. Where two components are co-located on a device, the computer-readable medium 152 can include a bus or other data conduit or plane. Where a first component is co-located on one device and a second component is located on a different device, the computer-readable medium 152 can include a wireless or wired back-end network or LAN. The computer-readable medium 152 can also encompass a relevant portion of a WAN or other network, if applicable.

The medical computer system 154 may include a computer system configured to input, access, modify, and save information relating to a patient's health. The medical computer system 154 may include memory, one or more processors, and/or sensors to scan, sense, detect, and/or record parameters relating to a patient's health.

The display system 156 may include a computer system configured to display patient health information. The display system 156 may include memory, one or more processors, and a display device. The display system 156 may be implemented as part of a computer system, a display of a wearable device, etc.

The patient identification system 158 may include a computer system configured to facilitate collecting, distributing, analyzing, and organizing patient data for clinical trial selection, placement, and enrollment. As noted herein, the patient identification system 158 may be configured to access patient information from a patient information database to provide tools for a medical practitioner to recommend or enroll patients for placement in clinical trials. For example, in the field of ophthalmology, the patient identification system may be configured to access patient data from the IRIS registry, which is a comprehensive eye disease clinical registry. The patient information database can include a trove of information relating to the patient including patient health/disease state, physician notes, and billing codes relating to disease diagnosis and treatment. The patient identification system 158 may include clinical trial engine(s) 160, patient selection engine(s) 162, future eligibility engine(s) 164, and patient follow-up engine(s) 166. One or more of the modules of the patient identification system 158 may be coupled to each other or to modules not shown.

As used herein, any “engine” may include one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors or a first engine and a second engine can share one or more processors with one another or other engines. Depending upon implementation-specific or other considerations, an engine can be centralized or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

As used herein, “datastores” may include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

The clinical trial engine(s) 160 may implement one or more automated agents configured to access and review ongoing and upcoming clinical trials. The clinical trial engine(s) 160 may access databases with patient information and be configured to review and display available clinical trials. In some implementations, the clinical trial engine(s) 160 access and display general information relating to ongoing and upcoming clinical trials, including a brief description of clinical trials, and the timeline for enrollment and participating, and eligibility requirements. The clinical trial engine(s) 160 may also be configured provide additional detailed information about each study, including statistics for the study and a comparison between potential clinical trial sites. The clinical trial engine(s) 160 may further provide functionality for enrollment of new patients into current and upcoming clinical trials. The clinical trial engine(s) 160 may provide patient data, clinical trial data, and/or other data to other modules of the patient identification system 158.

For example, the eligibility requirements for a clinical trial may include disease type, disease progression, symptoms, treatment type, age, gender, geographic location, proximity to clinical sites, and outcomes. Eligibility requirements may additional require specific diseases or symptoms. As one example, in the field of ophthalmology, the eligibility requirements may specify required ranges for visual acuity, the presence of neovascularization, fluid, or hemorrhage under the fovea, OCT of the macula, choroidal neovascularization, Drusen in either eye or late AMD in either eye, etc. As a further example in the field of ophthalmology, the eligibility requirements may require or prohibit certain types of treatment (i.e., prior treatment for prophylactics, condition preventing a certain time period of follow up, prior treatment with IV bevacizumab, the use of investigational therapeutics likely to affect the eye, current systemic anti-VEGF therapies, or other contraindications to the study drug).

The patient selection engine(s) 162 may implement one or more automated agents configured to access patient data, review patient information, and enroll patients in ongoing and upcoming clinical trials. The patient selection engine(s) 162 may provide access to patient information databases to access and review patients that may be eligible for enrollment in ongoing and upcoming clinical trials. In some implementations, the patient selection engine(s) 162 access and display lists of potential patients that has been generated for that clinical site based on how well the patients fit the needs of the trial. The patient selection engine(s) 162 may also be configured provide additional detailed information about each patient, including why the selected patients are good fits for a particular study and additional information that is required for patient enrollment. The patient selection engine(s) 162 may further provide the functionality for a registered physician to review a list of potential patients, review the requirements of the clinical trial, and approve the enrollment of those patients in an ongoing or upcoming clinical trial. The patient selection engine(s) 162 may provide patient data, clinical trial data, and/or other data to other modules of the patient identification system 158.

The future eligibility engine(s) 164 may implement one or more automated agents configured to predict whether a patient that is not currently eligible for enrollment in a clinical trial may be eligible for the trial in the future, particularly prior to the start date of the clinical trial. In some implementations, the machine learning engine(s) 164 acquire clinical trial data, patient data, and historical patient data from the clinical trial engine(s) 160 and the patient selection engine(s) 162 and apply machine learning algorithms to predict a clinical trial future eligibility score. In some implementations, the machine learning engine(s) 164 use a trained convolutional neural network and/or trained classifiers to classify a patient into one or more identified categories of likely to be eligible for a clinical trial, likely to be ineligible for a clinical trial, etc. Examples of machine learning systems implemented by the machine learning engine(s) 164 may include Decision Tree, Random Forest, Logistic Regression, Support Vector Machine, AdaBOOST, K-Nearest Neighbor (KNN), Quadratic Discriminant Analysis, Neural Network, etc., to determine a clinical trial future eligibility score. The predicted score can be incorporated into the patient information and/or clinical trial database(s).

The patient follow-up engine(s) 166 may be configured to store and/or provide instructions and functionality to contact and/or follow-up with patients who are potential clinical trial enrollees. The patient follow-up engine(s) 166 may provide alerts, reminders, and/or prompts to a medical practitioner with contact information for potential enrollees. The patient follow-up engine(s) 166 may further provide the ability to update patient data with information pertaining to discussions with the patient regarding clinical trials and enrollment.

FIG. 1B is a diagram showing an example of the clinical trial engine(s) 160a. The clinical trial engine(s) 160a may include a trial information engine 168, a trial enrollment engine 170, and a clinical trial datastores 172. One or more of the modules of the clinical trial engine(s) 160a may be coupled to each other or to modules not shown.

The trial information engine 168 may implement one or more automated agents configured to access, sort, and display information pertaining to ongoing and upcoming clinical trials. The trial information engine 168 may implement one or more automated agents configured to display a list of current case studies, including a brief description, and a timeline for enrollment and completion of the study. The trial information engine 168 may further implement one or more automated agents configured to display detailed information about the study including statistics and metrics comparing where a particular clinical site stands compared to other potential or ongoing clinical sites.

The trial enrollment engine 170 may implement one or more automated agents configured to enroll eligible patients in ongoing and upcoming clinical trials. The trial enrollment engine 170 may implement one or more automated agents configured to display a list of current case studies seeking new patients, input eligible patient data, and enroll new patients in case studies. The trial enrollment engine 170 may implement one or more automated agents configured to confirm enrollment of new patients in case studies and provide information about enrollment and how to participate in the study.

Clinical trial datastore 172 may implement one or more automated agents configured to store information pertaining to clinical trials, clinical trial enrollment requirements, statistics, information about clinical trial sites, and may further be configured to update clinical trials information when new patients are enrolled.

FIG. 1C is a diagram showing an example of the patient selection engine(s) 162a. The patient selection engine(s) 162a may include a patient information engine 174, a patient review engine 176, and a patient datastores 178. One or more of the modules of the patient selection engine(s) 160a may be coupled to each other or to modules not shown.

The patient information engine 174 may implement one or more automated agents configured to access, sort, and display patient health data pertaining to patients that may be eligible for enrollment and participation in ongoing and upcoming clinical trials. The patient information engine 174 may implement one or more automated agents configured to display a list of potential patients that has been generated for that clinical site. The list of potential patients can be a subset of all patients that receive medical care at the clinical site. In one example, the patient information engine 174 can implement one or more automated agents to automatically determine patients that are eligible for a clinical trial based on the patient health data and the eligibility requirements of the clinical trial. In some examples, the patient information engine 174 can assign an eligibility score to each patient based on how many eligibility requirements the patient satisfies. A subset of patients may be selected based on the eligibility score. The patient information engine 174 may further implement one or more automated agents configured to display detailed information about the patient, including why the selected patients are good fits for a particular study and additional information that is required for patient enrollment.

The patient health data may comprise any information relating to the patient including patient health, disease state, age, gender, geographic location, treatment type, symptoms, disease progression, other relevant diseases, physician notes, and billing codes relating to disease diagnosis and treatment. As one example, in the field of ophthalmology, the patient health data may include ranges for visual acuity, the presence of neovascularization, fluid, or hemorrhage under the fovea, OCT of the macula, choroidal neovascularization, Drusen in either eye or late AMD in either eye, etc. As a further example in the field of ophthalmology, the patient health data may include specific details on the type of treatment or lack thereof (i.e., prior treatment for prophylactics, condition preventing a certain time period of follow up, prior treatment with IV bevacizumab, the use of investigational therapeutics likely to affect the eye, current systemic anti-VEGF therapies, or other contraindications to the study drug).

The patient review engine 176 may implement one or more automated agents configured to allow a registered physician or other medical practitioner to review the list of potential patients from a clinical site coordinator. The patient review engine 176 may implement one or more automated agents configured to display the list of potential patients, the requirements of the clinical trial, and any other information pertinent to the potential enrollment of the patents in that clinical trial. The patient review engine 176 may implement one or more automated agents configured to allow the physician or medical practitioner to confirm and approve the potential enrollment of one or more patients in the clinical study.

Patient datastores 178 may implement one or more automated agents configured to store information pertaining to patient medical data, and may further be configured to update patient data when new patients are approved, enrolled, and selected for clinical trials. Patient datastores 178 may further store historical patient data, including data pertaining to prior eligibility of patients for clinical trials, and information relating to treatment and/or progression of diseased states.

FIG. 1D is a diagram showing an example of future eligibility engine(s) 164a. The future eligibility engine(s) 164a may acquire clinical trial data, patient data, and historical patient data from the clinical trial engine(s) 160 and the patient selection engine(s) 162. The of future eligibility engine(s) 164a may include a machine learning engine 180 and a future eligibility datastore 182.

The machine learning engine 180 may implement one or more automated agents configured to use machine learning techniques to classify the eligibility of a patient for a future clinical trial. In some implementations, the machine learning engine 180 may acquire clinical trial data, patient data, and historical patient data from the clinical trial engine(s) 160 and the patient selection engine(s) 162. Using a trained classifier, the machine learning engine 180 may provide an identifier (e.g., a statistical or other score) that determines if a patient that is not currently eligible for a clinical trial may be eligible in the future. As examples, the machine learning engine 180 may use a classifier trained to correlate the patient's current health and treatment regimen with historical data regarding the progression of disease based on that treatment to determine if the patient's disease may progress to a state in the future that would qualify the patient for clinical trial enrollment. The machine learning engine 180 may incorporate one or more machine learning techniques. Examples of such techniques include Convolutional Neural Networks (CNN), Decision Tree, Random Forest, Logistic Regression, Support Vector Machine, AdaBOOST, K-Nearest Neighbor (KNN), Quadratic Discriminant Analysis, Neural Network, etc. The machine learning engine 180 can provide an output with a clinical trial future eligibility score. The output can be, for example, a binary score or a linear score.

The future eligibility datastore 182 may be configured to store data relating to the clinical trial future eligibility score from the machine learning engine 180 (e.g., the output from the machine learning engine). In some implementations, the clinical trial future eligibility score is a binary labeling score (e.g., a score of 1 for a patient that is likely to be eligible for a clinical trial in the future and a score of 0 for a patient that is not likely to be eligible for a clinical trial in the future). In other implementations, the clinical trial future eligibility score is a linear labeling score (e.g., a score ranging from 0 to 10, where 0 indicates that a patient is unlikely to qualify for a clinical trial and a score of 10 indicates that a patient is likely to qualify for a clinical trial).

FIG. 1E is a diagram showing an example of patient follow-up engine(s) 166a. The patient follow-up engine(s) 166a may be configured to store and/or provide instructions and functionality to contact and/or follow-up with patients who are potential clinical trial enrollees. The patient follow-up engine(s) 166a may provide alerts, reminders, and/or prompts to a medical practitioner with contact information for potential enrollees. The patient follow-up engine(s) 166a may further provide the ability to update patient data with information pertaining to discussions with the patient regarding clinical trials and enrollment.

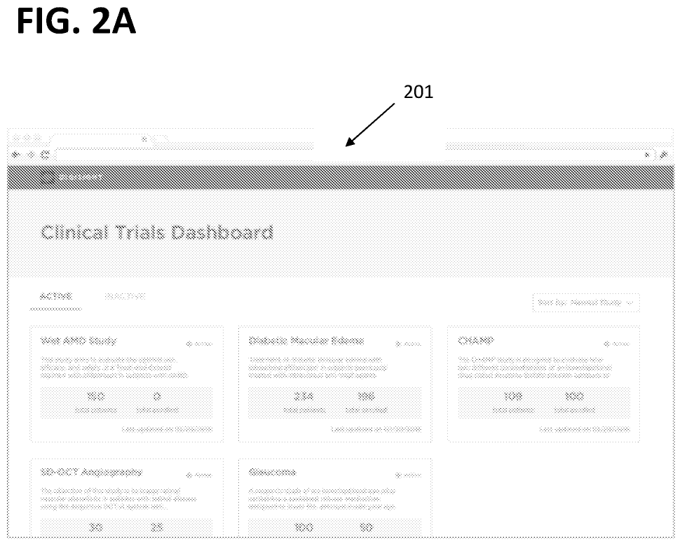

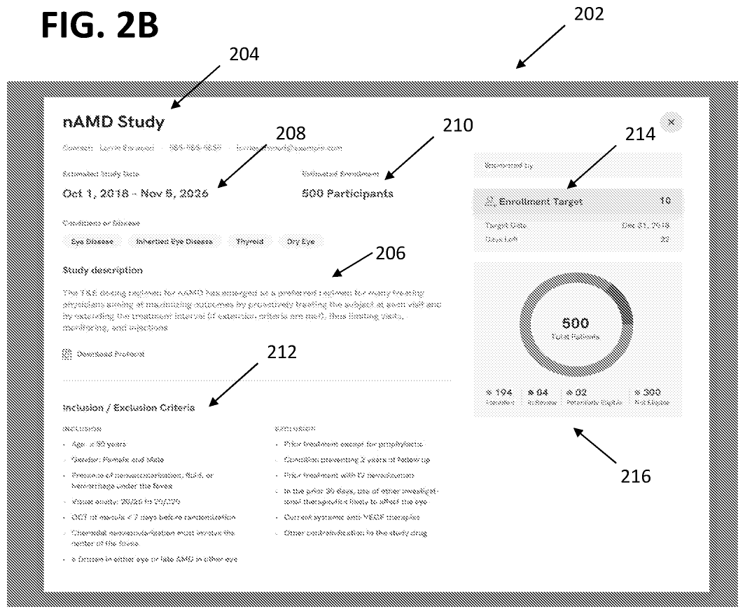

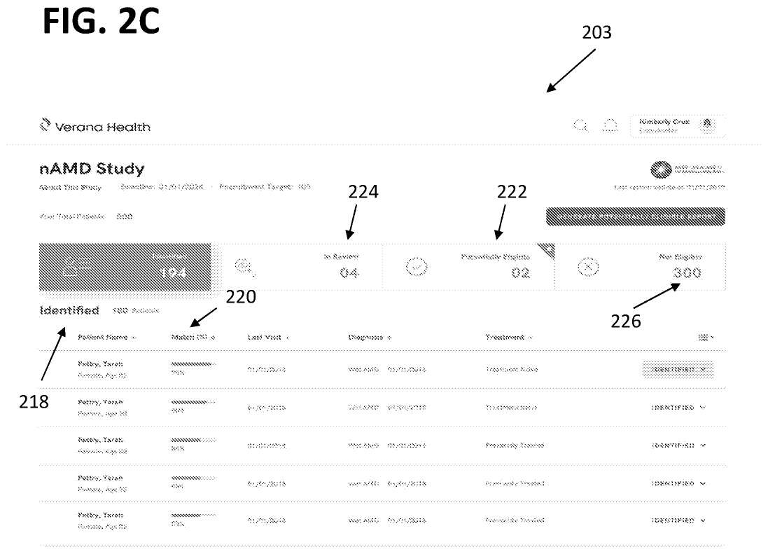

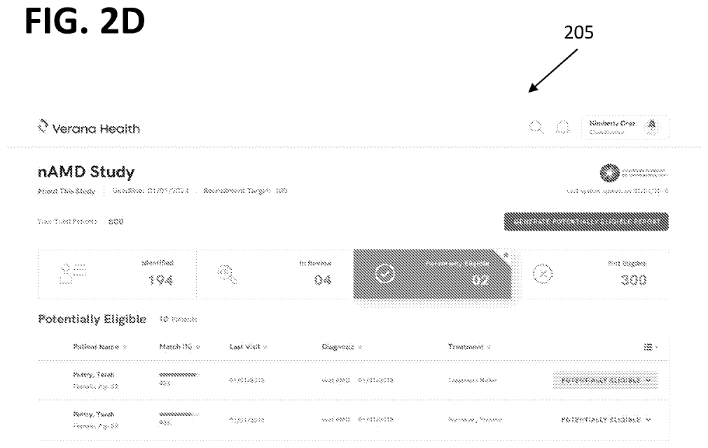

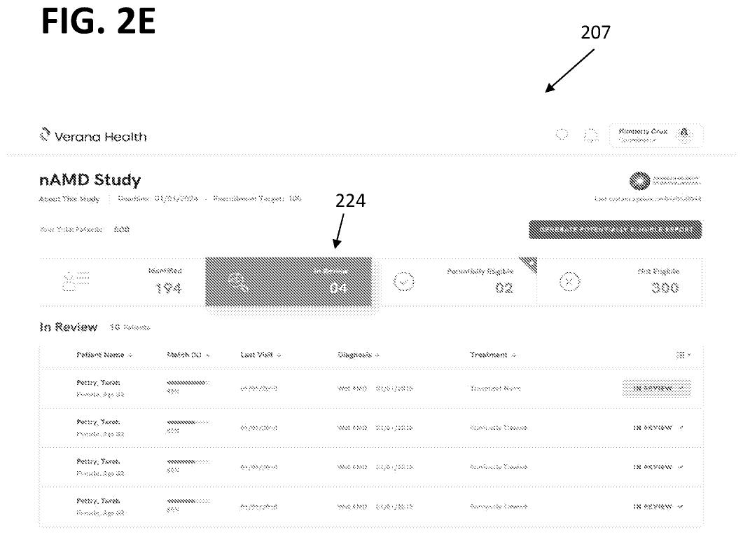

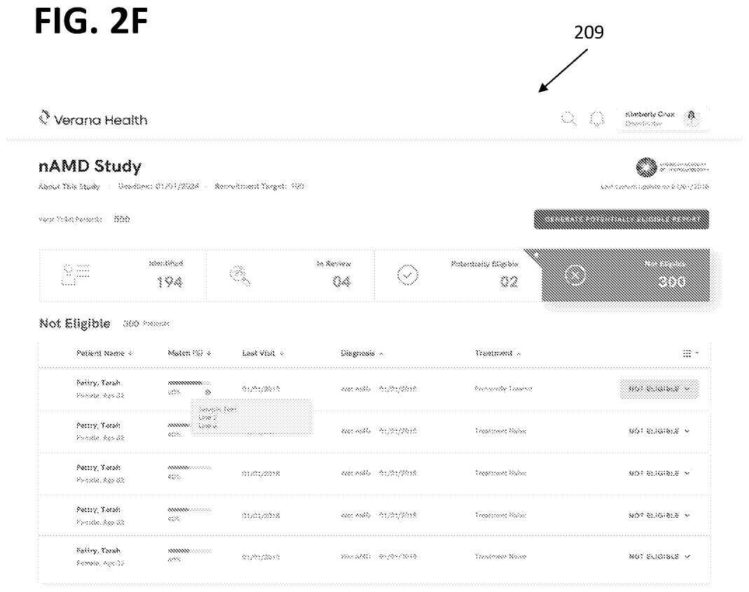

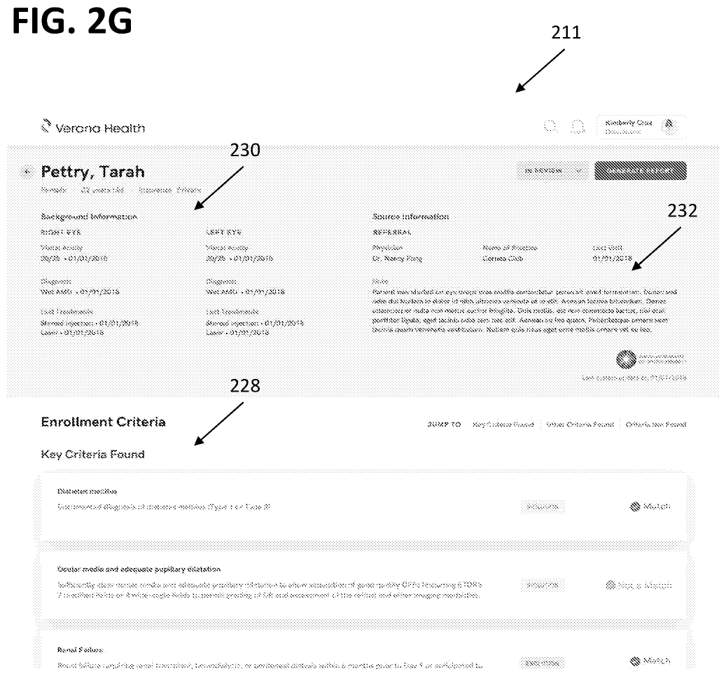

FIGS. 2A-2G illustrate examples of exemplary data visualization for a clinical trial coordinator at a specific medical practice enabled by accessing the medical practice specific data. FIG. 2A represents a screen view 201 for all clinical trials being performed at the specific site. The clinical coordinator can view active studies, as well as completed or upcoming studies from this screen view.

FIG. 2B represents a screen view 202 that provides site statistics for a particular study to help the site to keep track of their patient recruitment operations. The sponsor provided clinical study protocol can also be viewed and downloaded from this screen view. In this way a clinical coordinator may see all trial activity irrespective of sponsor organization or other entities involved. In screen view 202 of FIG. 2B, a clinical coordinator may the name of the current study 204, including a brief description 206 of the study, an estimated timeline 208 for the study, the estimated enrollment 210, and the inclusion/exclusion criteria 212 for eligibility in the study. The screen view 202 can also provide the clinical coordinator with an enrollment target 214 for that specific site, including detailed statistics 216 about the patient population that has been identified as potentially eligible for the study.

FIG. 2C represents screen view 203 which allows a clinical coordinator to see a list 218 of potentially eligible patients that has been generated for a particular clinical site. The screen view can include a score or match percentage (%) 220 to indicate how well a patient matches the eligibility criteria provided by the study sponsor. The clinical coordinator can search for patients by first or last name, and can sort columns of the list to view by patient names, recruitment status, or match percentage. The screen view 203 can further display statistics comparing the present site to other clinical sites by seeing the number of potential patients.

Additionally, the screen view 204 can also indicate patients that are not currently eligible for the study, but may potentially be eligible 222 in the future (i.e., by the time the study starts). This feature corresponds to the processes of the future eligibility engine(s) 164 described above. The list of potential patients can include a prioritized list of patients based on how well they fit a particular study. The list of potentially eligible patients is shown in more detail in screen view 205 of FIG. 2D. Furthermore, screen view 203 of FIG. 2C allows a clinical coordinator to tag specific patients for further review 224 by a physician or medical practitioner, described in more detail below with respect to FIG. 2E. Furthermore, screen view 203 also shows a list of patients that are not eligible 226, shown in more detail in screen view 209 of FIG. 2F. These patients can be deemed ineligible by any combination of the clinical coordinator, physician, or software.

FIG. 2E represents screen view 207, which allows the clinical coordinator to identify specific patients for further review by a physician or licensed medical provider. These patients can be selected by the clinical trial coordinator as likely to be eligible for the clinical trial, based on their own knowledge and manual chart review. This list is also used in a workflow between the clinical trial coordinator and physician or medical provider to approve patients for next steps in the enrollment process.

FIG. 2G represents screen view 211 which allows a clinical coordinator to see a specific patient details, including details 228 of the patient's eligibility in a selected clinical trial, background information 230 on the patient, and source information including medical notes 232. The clinical coordinator can see why a particular patient is a good match for the clinical trial, and also see additional information that is needed to help identify if the patient is a good match. The details 228 of the patient's eligibility can include inclusion/exclusion criteria for the clinical trial and whether or not the patient matches that specific inclusion/exclusion criteria.

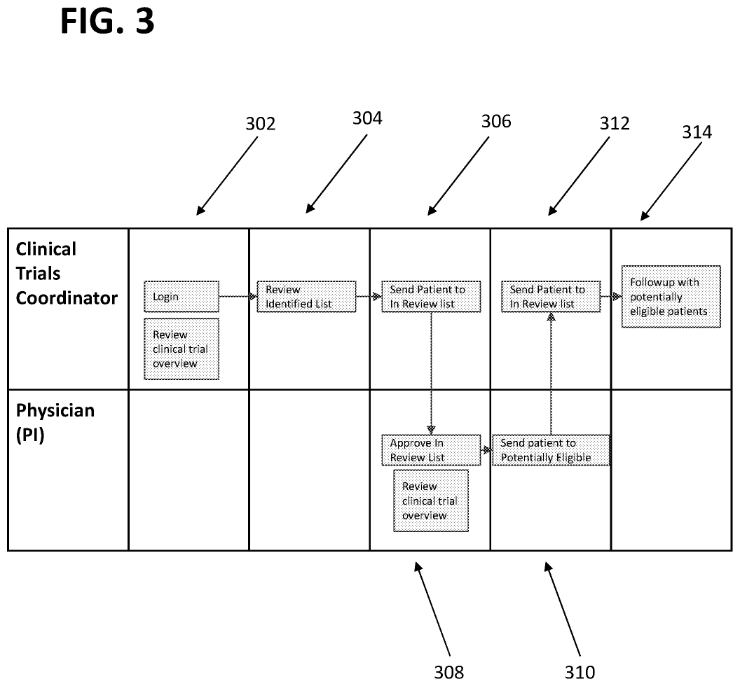

FIG. 3 is a flowchart illustrating the workflow from a clinical coordinator's perspective for collecting, distributing, analyzing, and organizing patient data for clinical trial selection, placement, and enrollment. This method may be automatically implemented by a system, such as one or more of the systems in the computing environment 100A, shown in FIG. 1A. At an operation 302, the system may automatically collect and display data pertaining to current, expired, or upcoming clinical trials data from one or more databases. The clinical trial data may be provided by a life sciences company, or acquired from a database that includes information on current, upcoming, or expired clinical trials. A clinical trials coordinator at a medical practice may access and view the clinical trials data at operation 302. The clinical trials data can include the name of the current study, including a brief description of the study, an estimated timeline for the study, the estimated enrollment, and the inclusion/exclusion criteria for eligibility in the study. The clinical coordinator may also view an enrollment target for that specific site, including detailed statistics about the patient population that has been identified as potentially eligible for the study. Operation 302 corresponds with FIGS. 2A-2B and the related description above.

At an operation 304, a clinical coordinator can view and review an automatically generated list of potential patients for a selected clinical trial. The list of potential patients can include a score or match percentage (%) to indicate how well a patient matches the eligibility criteria provided by the study sponsor. The clinical coordinator can search for patients by first or last name, and can sort columns of the list to view by patient names, recruitment status, or match percentage. The clinical coordinator can further view statistics comparing the present site to other clinical sites by seeing the number of potential patients. Additionally, the clinical coordinator can view patients that are not currently eligible for the study, but may potentially be eligible in the future (i.e., by the time the study starts). Operation 304 corresponds to the processes of the future eligibility engine(s) 164 in FIGS. 1A and 1D, as well as FIGS. 2C and 2D as described above. The list of potential patients can include a prioritized list of patients based on how well they fit a particular study.

At operations 306 and 308, the clinical coordinator can send a list of potential patients to a physician or licensed medical practitioner for final review to be included in a clinical study. These patients can be selected by the clinical trial coordinator as likely to be eligible for the clinical trial, based on their own knowledge and manual chart review. This list is also used in a workflow between the clinical trial coordinator and physician or medical provider to approve patients for next steps in the enrollment process. Operations 306 and 308 correspond to FIGS. 2E and 2G as described above.

At operations 310 to 314, the clinical coordinator can receive the approved list of patients from the physician or licensed medical practitioner, and follow up with the patients to proceed with the enrollment process.

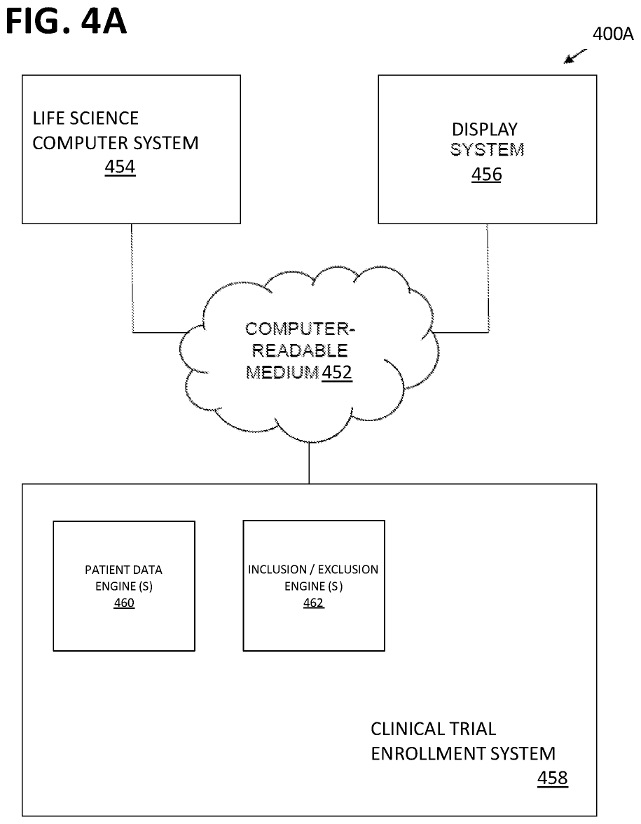

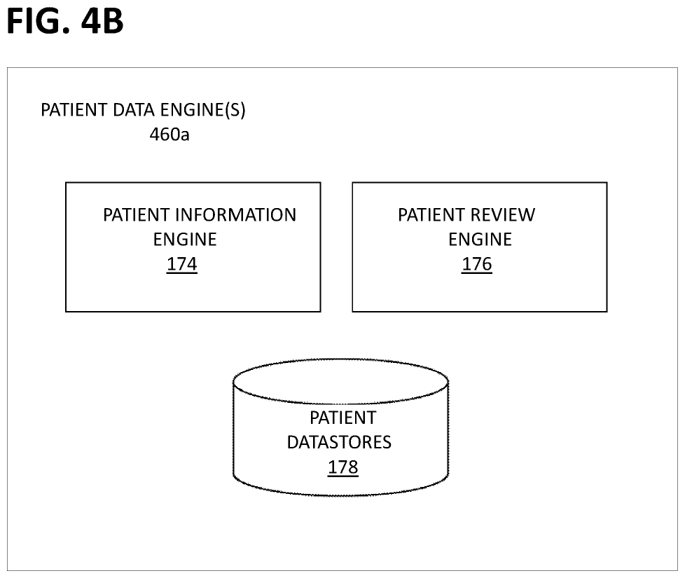

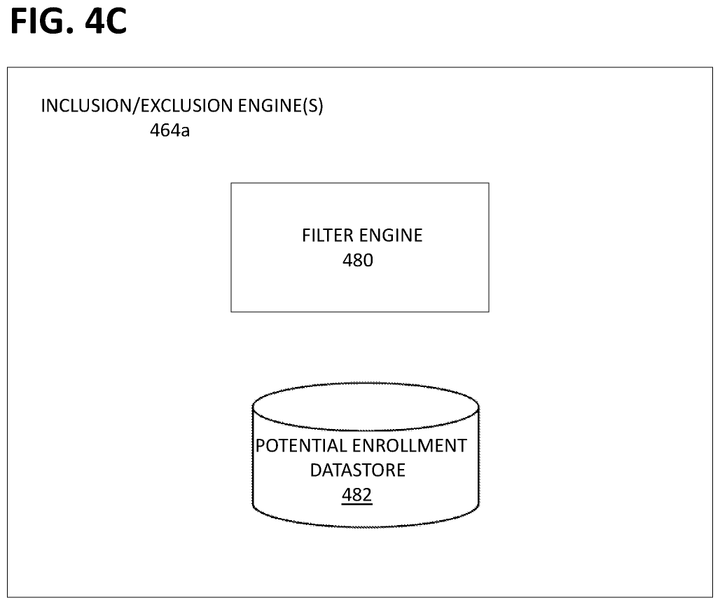

FIG. 4A is a diagram showing an example of a computing environment 400A configured to facilitate collecting, analyzing, and reviewing patient data for clinical trial selection, placement, and enrollment. The environment 400A includes a computer-readable medium 452, a life science computer system 454, a display system 456, and a patient clinical trial enrollment system 458. One or more of the modules in the computing environment 400A may be coupled to one another or to modules not explicitly shown.

The computer-readable medium 452 and other computer readable media discussed herein are intended to represent a variety of potentially applicable technologies. For example, the computer-readable medium 452 can be used to form a network or part of a network. Where two components are co-located on a device, the computer-readable medium 452 can include a bus or other data conduit or plane. Where a first component is co-located on one device and a second component is located on a different device, the computer-readable medium 452 can include a wireless or wired back-end network or LAN. The computer-readable medium 452 can also encompass a relevant portion of a WAN or other network, if applicable.

The life science computer system 454 may include a computer system configured to input, access, modify, and save information relating to a patient's health. The medical computer system 454 may include memory, one or more processors, and/or sensors to scan, sense, detect, and/or record parameters relating to a patient's health.

The display system 456 may include a computer system configured to display patient health information. The display system 456 may include memory, one or more processors, and a display device. The display system 456 may be implemented as part of a computer system, a display of a wearable device, etc.

The clinical trial enrollment system 458 may include a computer system configured to facilitate collecting, analyzing, and reviewing patient data for clinical trial selection, placement, and enrollment in one or more clinical trials. As noted herein, the clinical trial enrollment system 458 may be configured to access patient health data from a patient information database to provide tools for a life science company or clinical trial sponsor to find eligible patients for placement and enrollment in clinical trials. For example, in the field of ophthalmology, the clinical trial enrollment system may be configured to access patient data from the IRIS registry, which is a comprehensive eye disease clinical registry. Clinical trial enrollment system 458 may include patient data engine(s) 460 and inclusion/exclusion engine(s) 462. One or more of the modules of the clinical trial enrollment system 458 may be coupled to each other or to modules not shown.

The patient health data may comprise any information relating to the patient including patient health, disease state, age, gender, geographic location, treatment type, symptoms, disease progression, other relevant diseases, physician notes, and billing codes relating to disease diagnosis and treatment. As one example, in the field of ophthalmology, the patient health data may include ranges for visual acuity, the presence of neovascularization, fluid, or hemorrhage under the fovea, OCT of the macula, choroidal neovascularization, Drusen in either eye or late AMD in either eye, etc. As a further example in the field of ophthalmology, the patient health data may include specific details on the type of treatment or lack thereof (i.e., prior treatment for prophylactics, condition preventing a certain time period of follow up, prior treatment with IV bevacizumab, the use of investigational therapeutics likely to affect the eye, current systemic anti-VEGF therapies, or other contraindications to the study drug).

The patient data engine(s) 460 may implement one or more automated agents configured to access patient data, review patient information, and review the locations and concentrations of patients that may be eligible for enrollment in ongoing and upcoming clinical trials. The patient data engine(s) 460 may provide access to patient information databases to access and review patients that may be eligible for enrollment in ongoing and upcoming clinical trials. In some implementations, the patient data engine(s) 460 access and display lists and/or graphical maps of potential patients that has been generated for the user based on how well the patients fit the needs of the trial. The patient data engine(s) 460 may also be configured provide details on where patients with a particular disease or condition are located geographically.

The inclusion/exclusion engine(s) 462 may implement one or more automated agents configured to access patient data from the patient data engine(s) 460. The inclusion/exclusion engine(s) 462 may provide a user with automated agents and tools to filter and/or narrow or expand a potential patient pool from the patient data. In one implementation, the inclusion/exclusion engine(s) 462 provides customizable inclusion/exclusion criteria which can be used to identify patients from the patient data that are eligible for a current or upcoming clinical trial. For example, inclusion/exclusion criteria can include filtering a patient population by disease type, disease progression, symptoms, treatment type, age, gender, geographic location, proximity to clinical sites, and outcomes. Additional inclusion/exclusion criteria can be implemented related to a specific target disease. For example, a clinical trial seeking patients in the field of ophthalmology may additionally filter by visual acuity, intraocular pressure, laterality (left eye, right eye, both, study eye), intravitreal injection, laser treatments, coding systems (CPT, ICD10, ICD9, SNOMED, etc.), etc. The inclusion/exclusion engine(s) 462 can coordinate and communicate with the patient data engine(s) 460 to display and produce detailed maps of potential patients that satisfy the selected inclusion/exclusion criteria.

FIG. 4B is a diagram showing an example of the patient data engine(s) 460a. The patient data engine(s) 460a may include a patient information engine 474, a patient review engine 476, and patient datastores 478. One or more of the modules of the patient data engine(s) 460a may be coupled to each other or to modules not shown.

The patient information engine 474 may implement one or more automated agents configured to access, sort, and display information pertaining to patients that may be eligible for enrollment and participation in ongoing and upcoming clinical trials. The patient information engine 474 may implement one or more automated agents configured to display a list of potential patients that has been generated for a clinical trial. The patient information engine 474 may further implement one or more automated agents configured to display detailed information about the patient, including why the selected patients are good fits for a particular study and additional information that is required for patient enrollment.

The patient review engine 476 may implement one or more automated agents configured to allow a life science company or study sponsor to review the list of potential patients. The patient review engine 476 may implement one or more automated agents configured to display the list of potential patients, location densities of potential patients on a graphical map, the location of clinical sites with respect to potential patient populations, etc.

Patient datastores 478 may implement one or more automated agents configured to store information pertaining to patient medical data, and may further be configured to update patient data when new patients are identified, enrolled, and selected for clinical trials. Patient datastores 478 may further store historical patient data, including data pertaining to prior eligibility of patients for clinical trials, and information relating to treatment and/or progression of diseased states.

FIG. 4C is a diagram showing an example of the inclusion/exclusion engine(s) 462a which may include filter engine 480 and potential enrollment datastore 482. One or more of the modules of the inclusion/exclusion engine(s) 462a may be coupled to each other or to modules not shown.

The filter engine 480 may implement one or more automated agents configured to filter and/or narrow or expand a potential patient pool from the patient data. In one implementation, the filter engine 480 provides customizable inclusion/exclusion criteria which can be used to identify patients from the patient data that are eligible for a current or upcoming clinical trial. For example, inclusion/exclusion criteria can include filtering a patient population by disease type, disease progression, treatment type, age, gender, geographic location, proximity to clinical sites, outcomes, and even key word searches from within medical/doctor notes in the patient's medical file. The filter engine can coordinate and communicate with the patient information engine 478 to display and produce detailed maps of potential patients that satisfy the selected inclusion/exclusion criteria.

Potential enrollment datastores 482 may implement one or more automated agents configured to store information pertaining to clinical trial enrollment, and may further be configured to update clinical trial data when new patients are identified, enrolled, and selected for clinical trials. Potential enrollment datastore 482 may further store historical clinical trial data, including data pertaining to locations or clinical sites that have a high percentage of eligible patients or patients who enroll successfully in clinical trials.

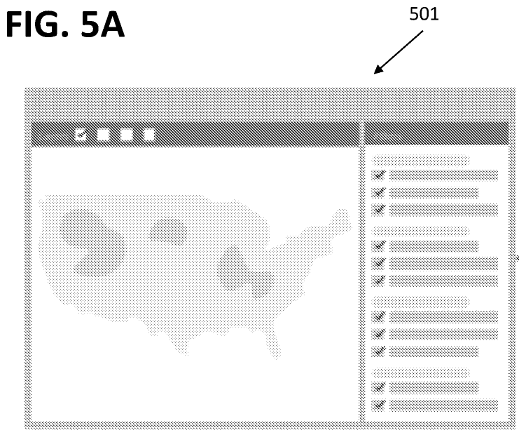

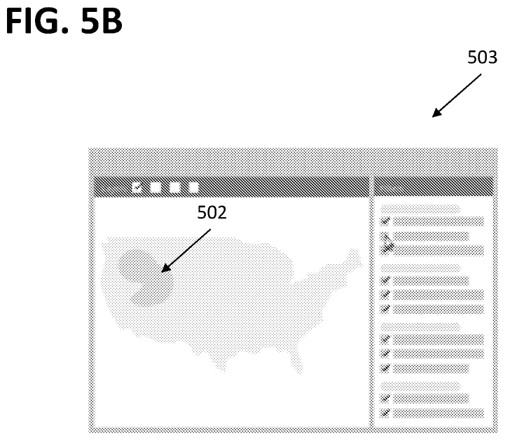

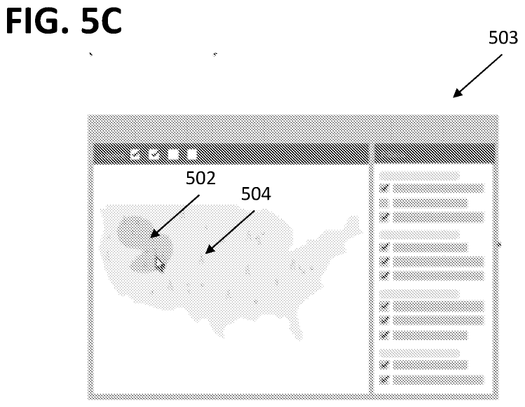

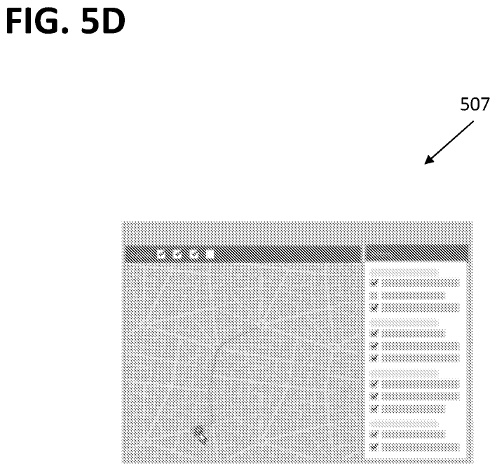

FIGS. 5A-5D are exemplary data visualization for a clinical program manager workflow enabled by accessing medical practice specific data. FIG. 5A is a view map 501 of a selected disease prevalence graphically displayed on a map of the United States. The selected disease can be chosen depending on the type of disease required for an upcoming clinical trial.

FIG. 5B is a view map 503 of the view in FIG. 5A after application of inclusion/exclusion criteria or filters for one or more patient factors. For example, inclusion/exclusion criteria or filters can include filtering a patient population by disease type, disease progression, treatment type, age, gender, geographic location, proximity to clinical sites, site type, previously used sites, clinical outcomes, and even key word searches from within medical/doctor notes in the patient's medical file. The view map 503 can visually illustrate a geographic location 502 that includes a plurality of potentially eligible patients for a specified clinical trial. FIG. 5C is a view map 505 that illustrates one or more clinical sites 504 located near the geographic location 502 of potentially eligible patients. FIG. 5D is a view map 507 that provides a zoomed-in view of a particular site, including more details about the site area and eligible nearby patients

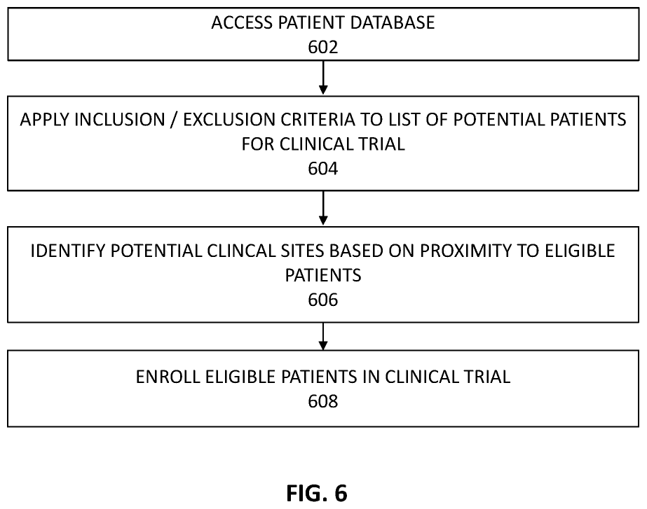

FIG. 6 is a flowchart illustrating the workflow from a life science company or study sponsor's perspective for reviewing, organizing, and filtering patient data for clinical trial selection, placement, and enrollment. This method may be automatically implemented by a system, such as one or more of the systems in the computing environment 400A, shown in FIG. 4A. At an operation 602, automated agents may access patient information from a patient information database to provide tools for the life science company or study sponsor to review or enroll patients for placement in clinical trials. For example, in the field of ophthalmology, the patient data may be accessed from the IRIS registry, which is a comprehensive eye disease clinical registry. The patient information database can include a trove of information relating to the patient including patient age, demographics, health/disease state, treatment, geographic location, physician notes, and billing codes relating to disease diagnosis and treatment.

At an operation 604, the automated agents may filter and/or narrow or expand a potential patient pool from the patient data. In one implementation, the inclusion/exclusion criteria can be used to identify patients from the patient data that are eligible for a current or upcoming clinical trial. For example, inclusion/exclusion criteria can include filtering a patient population by disease type, disease progression, treatment type, age, gender, geographic location, proximity to clinical sites, outcomes, and even key word searches from within medical/doctor notes in the patient's medical file.

At an operation 606, one or more automated agents may be configured to allow a life science company or study sponsor to identify a list of potential patients, location densities of potential patients on a graphical map, and the location of potential clinical sites with respect to high potential patient populations. This operation provides life science companies and/or study sponsors to identify clinical sites that are within proximity of the appropriately sized patient populations needed for a particular clinical trial.

At an operation 608, eligible patients can be enrolled in a clinical trial based on the results of steps 604 and 606 above.

Additional details of electronic medical records, medical information data collection and utilization and additional details of clinical trial management and processes may be appreciated by reference to U.S. Pat. Nos. 5,911,687; 5,619,991; 5,991,731; U.S. Pat. No. 7,054,823; United States Patent Application Publication US 2014/0249845; U.S. Pat. Nos. 9,740,831; 9,600,637, each of which is incorporated herein by reference in its entirety.

In one aspect, embodiments of the systems and methods described herein may be used with advantage to provide in any combination one or more of: market insight products; market planning reports; RWE studies; white label companion diagnostic information (i.e., PAXOS based information); salesforce effectiveness; salesforce territory design; label expansion; patient identification; patient screening; site identification; patient enrollment; and patient monitoring.

In one aspect, embodiments of the systems and methods described herein may be used with advantage for using data from IRIS Registry to address topics such as, without limitation, market share, business insights and characterizations of the standard of care. Similar analysis and tools may be providing using clinically specific data collected from specific medical practice groups from other medical practice affiliations as detailed above.

In one aspect, embodiments of the systems and methods described herein may be used with advantage in the field of clinical trials by reducing spend and time to market. In one representative embodiment, there is a method of improving a clinical trial utilizing computer implemented rules and processes for identifying clinical sites across the United States within a specific medical practice group. Next, additionally or optionally, there are steps for finding, screening, enrolling, and retaining patients that match clinical trial inclusion or clinical trial exclusion criteria. Next, additionally or optionally, there are steps for optimizing clinical trial design and implementing clinical trials with prevalence and outcomes data. Next, additionally or optionally, there is a step for simplifying quality reporting. And finally, additionally or optionally, there are steps for validating clinical trial results in a real world setting.

In one aspect, embodiments of the systems and methods described herein may be used with advantage in the field of market research to further assist in product or service development or for clinical trial assessment. In one representative embodiment, there is a method of providing market research insights utilizing computer implemented rules and processes within a data set related to a specific medical practice group. Next, additionally or optionally, there are steps for identifying unmet clinical needs within the specific medical practice group. Next, additionally or optionally, there are steps for uncovering market opportunities that align with new market criteria. Next, additionally or optionally, there are steps for calculating real world prevalence rates. Next, additionally or optionally, there are steps for understanding and providing insights into practice patterns of the medical practices within the specific medical practice group. Next, additionally or optionally, there are steps for specific identification of risk factors related to the specific medical practice group. Next, additionally or optionally, there are steps for analyzing the data to identify insights for competitive intelligence related to the specific medical practice group.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of identifying patients for enrollment in a clinical trial, the method comprising the steps of:
acquiring, in a computing device, clinical trial data including eligibility requirements for the clinical trial;
acquiring, in the computing device, patient health data for a plurality of patients;
determining, in the computing device, a subset of patients from the plurality of patients that are eligible for the clinical trial based on the clinical trial data and the patient health data; and
outputting from the computing device the subset of patients.

2. The method of claim 1, wherein the acquiring clinical trial data step comprises acquiring clinical trial data from a remote database.

3. The method of claim 1, wherein the eligibility requirements comprise a disease type.

4. The method of claim 1, wherein the eligibility requirements comprise a treatment type.

5. The method of claim 1, wherein the eligibility requirements comprise a lack of treatment.

6. The method of claim 1, wherein the eligibility requirements comprise a specific symptom.

7. The method of claim 1, wherein the eligibility requirements are selected from the group consisting of disease type, disease progression, symptoms, treatment type, age, gender, geographic location, proximity to clinical sites, and outcomes.

8. The method of claim 1, wherein the acquiring patient health data step comprises acquiring patient health data from a remote database.

9. The method of claim 8, wherein the remote database comprises an IRIS registry.

10. The method of claim 1, wherein the patient health data comprises a disease state.

11. The method of claim 1, wherein the patient health data comprises gender.

12. The method of claim 1, wherein the patient health data comprises age.

13. The method of claim 1, wherein the patient health data comprises a treatment type.

14. The method of claim 1, wherein the patient health data is selected from the group consisting of patient health, disease state, age, gender, geographic location, treatment type, symptoms, disease progression, other relevant diseases, physician notes, and billing codes relating to disease diagnosis and treatment.

15. The method of claim 1, wherein the determining step further comprises assigning an eligibility score to each of the plurality of patients based on how many eligibility requirements are satisfied by each patient.

16. A non-transitory computing device readable medium having instructions stored thereon for identifying patients for enrollment in a clinical trial, wherein the instructions are executable by a processor to cause a computing device to:
acquire clinical trial data including eligibility requirements for the clinical trial;
acquire patient health data for a plurality of patients;
determine a subset of patients from the plurality of patients that are eligible for the clinical trial based on the clinical trial data and the patient health data; and
output the subset of patients.

17. The non-transitory computing device readable medium of claim 16, wherein the instructions are executable by the processor to cause the computing device to acquire clinical trial data from a remote database.

18. The non-transitory computing device readable medium of claim 16, wherein the eligibility requirements comprise a disease type.

19. The non-transitory computing device readable medium of claim 16, wherein the eligibility requirements comprise a treatment type.

20. The non-transitory computing device readable medium of claim 16, wherein the eligibility requirements comprise a lack of treatment.

21. The non-transitory computing device readable medium of claim 16, wherein the eligibility requirements comprise a specific symptom.

22. The non-transitory computing device readable medium of claim 16, wherein the eligibility requirements are selected from the group consisting of disease type, disease progression, symptoms, treatment type, age, gender, geographic location, proximity to clinical sites, and outcomes.

23. The non-transitory computing device readable medium of claim 16, wherein the instructions are executable by the processor to cause the computing device to acquire patient health data from a remote database.

24. The non-transitory computing device readable medium of claim 23, wherein the remote database comprises an IRIS registry.

25. The non-transitory computing device readable medium of claim 16, wherein the patient health data comprises a disease state.

26. The non-transitory computing device readable medium of claim 16, wherein the patient health data comprises gender.

27. The non-transitory computing device readable medium of claim 16, wherein the patient health data comprises age.

28. The non-transitory computing device readable medium of claim 16, wherein the patient health data comprises a treatment type.

29. The non-transitory computing device readable medium of claim 16, wherein the patient health data is selected from the group consisting of patient health, disease state, age, gender, geographic location, treatment type, symptoms, disease progression, other relevant diseases, physician notes, and billing codes relating to disease diagnosis and treatment.

30. The non-transitory computing device readable medium of claim 16, wherein the instructions are executable by the processor to cause the computing device to assign an eligibility score to each of the plurality of patients based on how many eligibility requirements are satisfied by each patient.

31. A method of identifying patients for enrollment in a clinical trial, the method comprising the steps of:
acquiring, in a computing device, clinical trial data including eligibility requirements and a start date for the clinical trial;
acquiring, in the computing device, patient health data for a plurality of patients;
applying the clinical trial data and the patient health data to a classifier of the computing device to identify a subset of patients from the plurality of patients that are not currently eligible for the clinical trial but will likely be eligible for the clinical trial in the future; and
outputting the subset of patients from the computing device.

32. The method of claim 31, wherein the acquiring clinical trial data step comprises acquiring clinical trial data from a remote database.

33. The method of claim 31, wherein the eligibility requirements comprise a disease type.

34. The method of claim 31, wherein the eligibility requirements comprise a treatment type.

35. The method of claim 31, wherein the eligibility requirements comprise a lack of treatment.

36. The method of claim 31, wherein the eligibility requirements comprise a specific symptom.

37. The method of claim 31, wherein the eligibility requirements are selected from the group consisting of disease type, disease progression, symptoms, treatment type, age, gender, geographic location, proximity to clinical sites, and outcomes.

38. The method of claim 31, wherein the acquiring patient health data step comprises acquiring patient health data from a remote database.

39. The method of claim 38, wherein the remote database comprises an IRIS registry.

40. The method of claim 31, wherein the patient health data comprises a disease state.

41. The method of claim 31, wherein the patient health data comprises gender.

42. The method of claim 31, wherein the patient health data comprises age.

43. The method of claim 31, wherein the patient health data comprises a treatment type.

44. The method of claim 31, wherein the patient health data is selected from the group consisting of patient health, disease state, age, gender, geographic location, treatment type, symptoms, disease progression, other relevant diseases, physician notes, and billing codes relating to disease diagnosis and treatment.

45. The method of claim 31, wherein each of the subset of patients are assigned a binary score indicating that a patient is likely to be eligible for the clinical trial in the future.

46. The method of claim 31, wherein each of the subset of patients are assigned a linear score indicating along a scale how likely a patient is likely to be eligible for the clinical trial in the future.

47. A non-transitory computing device readable medium having instructions stored thereon for identifying patients for enrollment in a clinical trial, wherein the instructions are executable by a processor to cause a computing device to:
acquire clinical trial data including eligibility requirements and a start date for the clinical trial;
acquire patient health data for a plurality of patients;
apply the clinical trial data and the patient health data to a classifier of the computing device to identify a subset of patients from the plurality of patients that are not currently eligible for the clinical trial but will likely be eligible for the clinical trial in the future; and
output the subset of patients.

48. The non-transitory computing device readable medium of claim 47, wherein the instructions are executable by the processor to cause the computing device to acquire clinical trial data from a remote database.

49. The non-transitory computing device readable medium of claim 47, wherein the eligibility requirements comprise a disease type.

50. The non-transitory computing device readable medium of claim 47, wherein the eligibility requirements comprise a treatment type.

51. The non-transitory computing device readable medium of claim 47, wherein the eligibility requirements comprise a lack of treatment.

52. The non-transitory computing device readable medium of claim 47, wherein the eligibility requirements comprise a specific symptom.

53. The non-transitory computing device readable medium of claim 47, wherein the eligibility requirements are selected from the group consisting of disease type, disease progression, symptoms, treatment type, age, gender, geographic location, proximity to clinical sites, and outcomes.

54. The non-transitory computing device readable medium of claim 47, wherein the instructions are executable by the processor to cause the computing device to acquire patient health data from a remote database.

55. The non-transitory computing device readable medium of claim 54, wherein the remote database comprises an IRIS registry.

56. The non-transitory computing device readable medium of claim 47, wherein the patient health data comprises a disease state.

57. The non-transitory computing device readable medium of claim 47, wherein the patient health data comprises gender.

58. The non-transitory computing device readable medium of claim 47, wherein the patient health data comprises age.

59. The non-transitory computing device readable medium of claim 47, wherein the patient health data comprises a treatment type.

60. The non-transitory computing device readable medium of claim 47, wherein the patient health data is selected from the group consisting of patient health, disease state, age, gender, geographic location, treatment type, symptoms, disease progression, other relevant diseases, physician notes, and billing codes relating to disease diagnosis and treatment.

61. The non-transitory computing device readable medium of claim 47, wherein each of the subset of patients are assigned a binary score indicating that a patient is likely to be eligible for the clinical trial in the future.

62. The non-transitory computing device readable medium of claim 47, wherein each of the subset of patients are assigned a linear score indicating along a scale how likely a patient is likely to be eligible for the clinical trial in the future.

63. A method of identifying patients for enrollment in a clinical trial, the method comprising the steps of:
receiving, in a computing device, inclusion/exclusion criteria for the clinical trial;
acquiring, in the computing device, patient health data for a plurality of patients;
determining, in the computing device, a list of patients that are eligible for the clinical trial;
determining, in the computing device, a geographic location that includes a plurality of eligible patients proximate to a clinical site; and
outputting from the computing device the geographic location, the plurality of eligible patients, and the clinical site.

64. The method of claim 63, wherein the receiving inclusion/exclusion criteria step comprises receiving inclusion/exclusion criteria from a user.

65. The method of claim 63, wherein the inclusion/exclusion criteria comprise a disease type.

66. The method of claim 63, wherein the inclusion/exclusion criteria comprise a treatment type.

67. The method of claim 63, wherein the inclusion/exclusion criteria comprise a lack of treatment.

68. The method of claim 63, wherein the inclusion/exclusion criteria comprise a specific symptom.

69. The method of claim 63, wherein the inclusion/exclusion criteria are selected from the group consisting of disease type, disease progression, symptoms, treatment type, age, gender, geographic location, proximity to clinical sites, and outcomes.

70. The method of claim 63, wherein the acquiring patient health data step comprises acquiring patient health data from a remote database.

71. The method of claim 70, wherein the remote database comprises an IRIS registry.

72. The method of claim 63, wherein the patient health data comprises a disease state.

73. The method of claim 63, wherein the patient health data comprises gender.

74. The method of claim 63, wherein the patient health data comprises age.

75. The method of claim 63, wherein the patient health data comprises a treatment type.

76. The method of claim 63, wherein the patient health data is selected from the group consisting of patient health, disease state, age, gender, geographic location, treatment type, symptoms, disease progression, other relevant diseases, physician notes, and billing codes relating to disease diagnosis and treatment.

77. The method of claim 1, wherein the determining step further comprises assigning an eligibility score to each of the plurality of patients based on how many inclusion/exclusion criteria are satisfied by each patient.

78. A non-transitory computing device readable medium having instructions stored thereon for identifying patients for enrollment in a clinical trial, wherein the instructions are executable by a processor to cause a computing device to:
receive inclusion/exclusion criteria for the clinical trial;
acquire patient health data for a plurality of patients;
determine a list of patients that are eligible for the clinical trial
determine a geographic location that includes a plurality of eligible patients proximate to a clinical site; and
output the geographic location, the plurality of eligible patients, and the clinical site.

79. The non-transitory computing device readable medium of claim 78, wherein the instructions are executable by the processor to cause the computing device to receive inclusion/exclusion criteria from a user.

80. The non-transitory computing device readable medium of claim 78, wherein the inclusion/exclusion criteria comprise a disease type.

81. The non-transitory computing device readable medium of claim 78, wherein the inclusion/exclusion criteria comprise a treatment type.

82. The non-transitory computing device readable medium of claim 78, wherein the inclusion/exclusion criteria comprise a lack of treatment.

83. The non-transitory computing device readable medium of claim 78, wherein the inclusion/exclusion criteria comprise a specific symptom.

84. The non-transitory computing device readable medium of claim 78, wherein the inclusion/exclusion criteria are selected from the group consisting of disease type, disease progression, symptoms, treatment type, age, gender, geographic location, proximity to clinical sites, and outcomes.

85. The non-transitory computing device readable medium of claim 78, wherein the instructions are executable by the processor to cause the computing device to acquire patient health data from a remote database.

86. The non-transitory computing device readable medium of claim 85, wherein the remote database comprises an IRIS registry.

87. The non-transitory computing device readable medium of claim 78, wherein the patient health data comprises a disease state.

88. The non-transitory computing device readable medium of claim 78, wherein the patient health data comprises gender.

89. The non-transitory computing device readable medium of claim 78, wherein the patient health data comprises age.

90. The non-transitory computing device readable medium of claim 78, wherein the patient health data comprises a treatment type.

91. The non-transitory computing device readable medium of claim 78, wherein the patient health data is selected from the group consisting of patient health, disease state, age, gender, geographic location, treatment type, symptoms, disease progression, other relevant diseases, physician notes, and billing codes relating to disease diagnosis and treatment.

92. The non-transitory computing device readable medium of claim 78, wherein the instructions are executable by the processor to cause the computing device to assign an eligibility score to each of the plurality of patients based on how many inclusion/exclusion criteria are satisfied by each patient.
